(19) United States
(12) Patent Application Publication
(10) Pub. No.: US 2014/0255298 A1
(43) Pub. Date: Sep. 11, 2014

(54) RADIOPAQUE MARKERS FOR IMPLANTABLE MEDICAL LEADS

(71) Applicant: MEDTRONIC, INC., Minneapolis, MN (US)

(72) Inventors: Mary L. Cole, Saint Paul, MN (US); Jonathan M. Edward, St. Louis Park, MN (US); Kevin R. Seifert, Forest Lake, MN (US)

(73) Assignee: Medtronic, Inc., Minneapolis, MN (US)

(21) Appl. No.: 13/790,527

(22) Filed: Mar. 8, 2013

(51) Int. Cl. A61K 51/12

(52) U.S. Cl. CPC A61K 51/1282
USPC 424/1.11

(57) ABSTRACT

A radiopaque marker may include a body formed of a polymer and being adapted to be disposed around a portion of an implantable medical lead and a symbol formed of at least a radiologically dense powder or liquid added to the body and designed to identify the implantable medical lead as being safe application of a medical procedure. In some instances, the symbol may be formed of a polymer mixed with the radiologically dense powder or liquid. The body may also be formed of a polymer mixed with a radiologically dense powder or liquid wherein the mixed polymer forming the symbol is radiologically denser than the mixed polymer forming the body.

### TECHNICAL FIELD

This disclosure relates generally to radiopaque markers for implantable medical leads.

### BACKGROUND

A wide variety of implantable medical systems that deliver a therapy or monitor a physiologic condition of a patient have been clinically implanted or proposed for clinical implantation in patients. The implantable medical system may include an implantable medical lead connected to an implantable medical device (IMD). For example, implantable leads are commonly connected to implantable pacemakers, defibrillators, cardioverters, or the like, to form an implantable cardiac system that provides electrical stimulation to the heart or sensing of electrical activity of the heart. The electrical stimulation pulses can be delivered to the heart and the sensed electrical signals can be sensed by electrodes disposed on the leads, e.g., typically near distal ends of the leads. In another example, implantable leads may be connected to neurostimulation devices or other implantable medical devices to provide stimulation to muscle or tissue to treat neurological conditions.

Patients that have implantable medical systems may benefit, or even require, various medical imaging procedures to obtain images of internal structures of the patient. One common medical imaging procedure is magnetic resonance imaging (MRI). MRI procedures may generate higher resolution and/or better contrast images (particularly of soft tissues) than other medical imaging techniques. MRI procedures also generate these images without delivering ionizing radiation to the body of the patient, and, as a result, MRI procedures may be repeated without exposing the patient to such radiation.

During an MRI procedure, the patient or a particular part of the patient's body is positioned within an MRI device. The MRI device generates a variety of magnetic and electromagnetic fields to obtain the images of the patient, including a static magnetic field, gradient magnetic fields, and radio frequency (RF) fields. The static magnetic field may be generated by a primary magnet within the MRI device and may be present prior to initiation of the MRI procedure. The gradient magnetic fields may be generated by electromagnets of the MRI device and may be present during the MRI procedure. The RF fields may be generated by transmitting/receiving coils of the MRI device and may be present during the MRI procedure.

Many implantable medical systems are often contraindicated for an MRI procedure because the various fields produced by the MRI device may have an effect on the operation of the implantable medical system. Patients with these contraindicated implantable medical systems are therefore generally recommended to not have MRI procedures. Other implantable medical systems have been designed and tested as safe for use during MRI procedures under certain conditions, e.g., with certain types of MRI devices, certain isocenter, maximum average SAR, or the like. Other implantable medical systems will likely be designed and tested as safe for use during MRI procedures without any condition requirements.

### SUMMARY

Radiopaque markers may be used to represent that an implanted lead and/or implantable medical system is suitable for a particular medical procedure, such as an MRI procedure. The radiopaque markers are visible on an X-ray or during fluoroscopy so that administering personnel can have a visual assurance that the lead is designed for safe application of the medical procedure of interest. The radiopaque marker may be added to the lead during or after implantation of the lead in various ways including suturing, gluing, crimping, or clamping a radiopaque tag to the lead or to the device. Thus, if an implantable medical lead is later determined to be MR-compatible, the radiopaque marker may be added, such as at device replacement, to identify that the lead is designed for safe application of the medical procedure of interest. This disclosure provides a number of different radiopaque markers suitable for such use.

In one example, the disclosure is directed to a radiopaque marker that includes a body formed of a polymer and being adapted to be disposed around a portion of an implantable medical lead and a symbol formed of at least a radiologically dense powder added to the body and designed to identify the implantable medical lead as being safe application of a medical procedure. In some instances, the symbol may be formed of a polymer mixed with the radiologically dense powder. The body may also be formed of a polymer mixed with a radiologically dense powder wherein the mixed polymer forming the symbol is radiologically denser than the mixed polymer forming the body.

In another example, the disclosure is directed to a radiopaque marker that includes a body formed of a polymer and being adapted to be disposed around a portion of an implantable medical lead and a symbol formed of at least a radiologically dense liquid added to the body and designed to identify the implantable medical lead as being safe application of a medical procedure.

In a further example, the disclosure is directed to a radiopaque marker that includes a body being adapted to be disposed around a portion of an implantable medical lead and formed from a polymer mixed with a radiopacifier. The polymer is designed to form a symbol that identifies the implantable medical lead as being designed for safe application of a medical procedure. In some instances, the body of the radiopaque marker includes portions of varying thicknesses, the thick portions of the body being designed to form the symbol that identifies the implantable medical lead as being designed for safe application of a medical procedure such that the thick portions of the body appear more radiologically dense during an imaging procedure. In other instances, the body of the radiopaque marker may have a relatively uniform thickness and is shaped into the symbol that identifies the implantable medical lead as being designed for safe application of a medical procedure.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF THE DRAWINGS

FIG. 1 is a conceptual diagram illustrating a magnetic resonance imaging (MRI) environment that includes an MRI device.

FIG. 2 is a conceptual diagram of an example implantable medical system that provides electrical stimulation therapy to a heart of a patient.

FIGS. 3A and 3B are schematic diagrams illustrating an example radiopaque marker that may be connected to implantable medical leads to identify the leads as being designed for safe application of a medical procedure.

FIGS. 4A and 4B are schematic diagrams illustrating an example radiopaque marker that may be connected to implantable medical leads to identify the leads as being designed for safe application of a medical procedure.

FIG. 5 is a schematic diagram illustrating an example radiopaque marker that may be connected to implantable medical leads to identify the leads as being designed for safe application of a medical procedure.

FIGS. 6A-C are schematic diagrams illustrating an example radiopaque marker that may be connected to implantable medical leads to identify the leads as being designed for safe application of a medical procedure.

FIGS. 7A and 7B are schematic diagrams illustrating an example radiopaque marker that may be connected to implantable medical leads to identify the leads as being designed for safe application of a medical procedure.

FIGS. 8A and 8B are schematic diagrams illustrating an example radiopaque marker that may be connected to implantable medical leads to identify the leads as being designed for safe application of a medical procedure.

### DETAILED DESCRIPTION

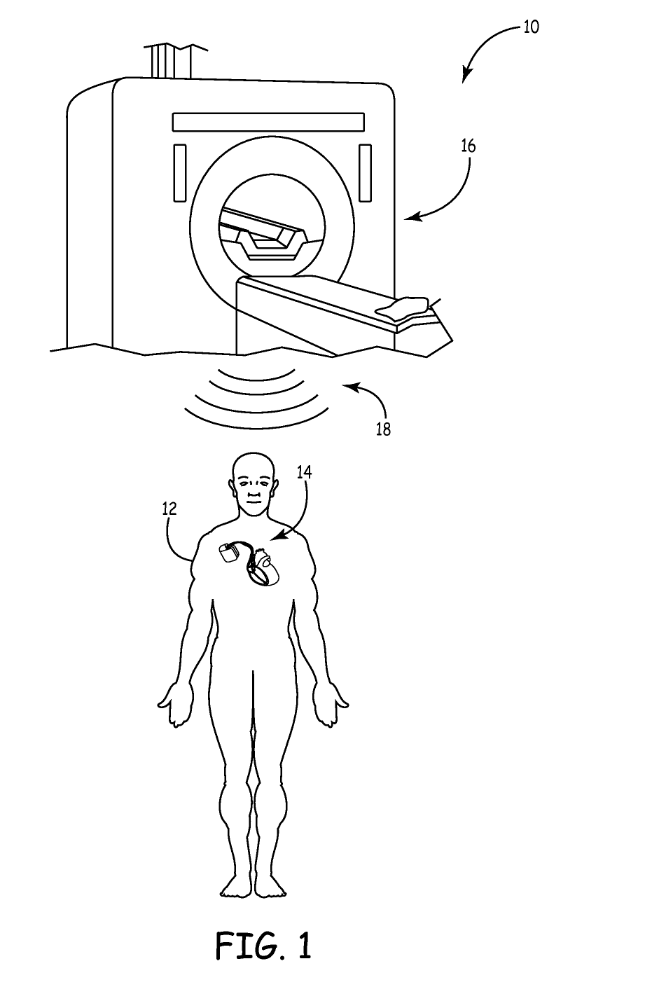

FIG. 1 is a conceptual diagram illustrating a magnetic resonance imaging (MRI) environment 10 that includes an MRI device 16. MRI device 16 may include a patient table on which patient 12 is placed prior to and during an MRI scan. The patient table is adjusted to position at least a portion of patient 12 within a bore of MRI device 16 (the “MRI bore”). While positioned within the MRI bore, patient 12 is subjected to a number of magnetic and RF fields to produce images of the portion of the body within the bore for diagnosing injuries, diseases, and/or disorders.

MRI device 16 includes a scanning portion that houses a primary magnet of MRI device 16 that generates a static MRI field. The static MRI field is a large non time-varying magnetic field that is typically always present around MRI device 16 whether or not an MRI scan is in progress. MRI device 16 also includes a plurality of gradient magnetic field coils that generate gradient magnetic fields. Gradient magnetic fields are pulsed magnetic fields that are typically only present while the MRI scan is in progress. MRI device 16 further includes one or more RF coils that generate RF fields. RF fields are pulsed high frequency fields that are also typically only present while the MRI scan is in progress.

The magnitude, frequency or other characteristic of the static MRI field, gradient magnetic fields and RF fields may vary based on the type of MRI device 16 producing the field or the type of MRI procedure being performed. A 1.5 T MRI device, for example, will produce a static magnetic field of approximately 1.5 Tesla and have a corresponding RF frequency of approximately 64 megahertz (MHz) while a 3.0 T MRI device will produce a static magnetic field of approximately 3.0 Tesla and have a corresponding RF frequency of approximately 128 MHz. However, other MRI devices may generate fields of different magnitude or frequency.

Although environment 10 is described as including an MRI device 16 that generates external fields 18, environment 10 may include sources of external fields 18 in addition to or instead of MRI device 16, such as devices used for electrocautery procedures, diathermy procedures, ablation procedures, electrical therapy procedures, magnetic therapy procedures or the like. Moreover, environment 10 may include non-medical sources of external fields 18, such as an interrogation unit of a radio frequency (RF) security gate.

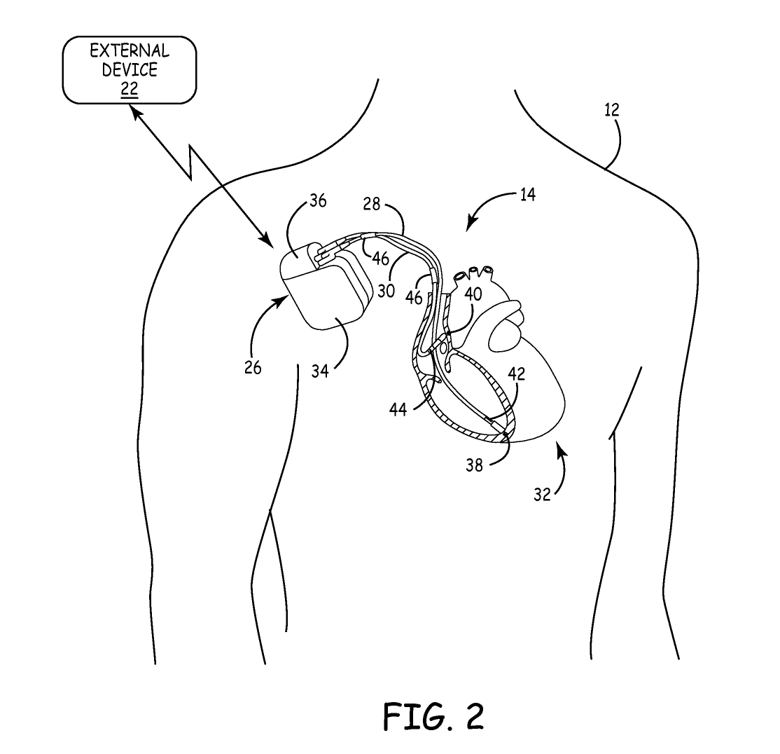

Implantable medical system 14 may, in one example, include an implantable medical device (IMD) connected to one or more leads. FIG. 2 is a schematic diagram illustrating implantable medical system 14 in further detail. Implantable medical system 14 includes an IMD 22 connected to leads 28 and 30. Implantable medical system 14 may be an implantable cardiac system that senses electrical activity of a heart and/or provides electrical stimulation therapy to the heart. Implantable medical system 14 may, for example, be an implantable pacemaker system, implantable cardioverter defibrillator (ICD) system, cardiac resynchronization therapy defibrillator (CRT-D) system, implantable cardioverter system, cardiac monitoring system, subcutaneous cardiac ICD system, or combinations thereof. Although illustrated in FIGS. 1 and 2 as an implantable cardiac system, implantable medical system 14 may alternatively be a non-cardiac implantable medical system, such as an implantable neurostimulation system with leads implanted within a brain, spine, or other location to provide electrical stimulation therapy to that location.

IMD 22 includes a housing 34 within which components of IMD 22 are housed. Housing 34 can be formed from conductive materials, non-conductive materials or a combination thereof. IMD 22 also includes a connector block 36 that includes electrical feedthroughs, through which electrical connections are made between conductors within leads 28 and 30 and electronic components included within housing 34. Housing 34 may house one or more processors, memories, transmitters, receivers, sensors, sensing circuitry, therapy circuitry, battery, and/or other appropriate components. Housing 34 is configured to be implanted in a patient, such as patient 12.

Leads 28 and 30 each include one or more electrodes. In the example illustrated in FIG. 2, leads 28 and 30 each include tip electrodes 38 and 40 and ring electrodes 42 and 44 located near a distal end of their respective leads 28 and 30. When implanted, tip electrodes 38 and 40 and/or ring electrodes 42 and 44 are placed relative to or in a selected tissue, muscle, nerve or other location within the patient 12. Although leads 28 and 30 are illustrated as including respective tip and ring electrodes, in other examples, one or both of leads 28 or 30 (or other lead) may include one or more than two electrodes. For example, a quadripolar lead may be provided that includes four electrodes (e.g., a hemispherical tip electrode and three ring electrodes or four ring-type electrodes) for use in multi-pole pacing applications.

In the example illustrated in FIG. 2, tip electrodes 38 and 40 are extendable helically shaped electrodes to facilitate fixation of the distal end of leads 28 and 30 to the target location within patient 12. In this manner, tip electrodes 38 and 40 are formed to define a fixation mechanism. In other embodiments, one or both of tip electrodes 38 and 40 may be formed to define fixation mechanisms of other structures. In other instances, leads 28 and 30 may include a fixation mechanism separate from tip electrode 38 and 40. For example, tip electrode 38 may take a different shape, such as a hemispherical electrode, and fixation mechanisms can be any appropriate type, including a grapple mechanism, a helical or screw mechanism, a drug-coated connection mechanism in which the drug(s) serves to reduce infection and/or swelling of the tissue, or other attachment mechanism.

One or more conductors (not shown in FIG. 2) extend within leads 28 and 30 from connector block 36 along the length of the lead to engage respective tip electrodes 38 and 40 and ring electrode 42 and 44. In this manner, each of electrodes 38, 40, 42 and 44 is electrically coupled to at least one respective conductor within its associated lead body. For example, a first electrical conductor can extend along the length of the body of lead 28 from connector block 36 and electrically couple to tip electrode 38 and a second electrical conductor can extend along the length of the body of lead 28 from connector block 36 and electrically couple to ring electrode 42. The respective conductors may electrically couple to circuitry, such as a therapy module or a sensing module, of IMD 22 via connections in connector block 36. The electrical conductors transmit therapy, e.g., pacing pulses or other stimulation, from the therapy module within IMD 22 to one or more of electrodes 38, 40, 42, and 44 and transmit sensed electrical signals from one or more of electrodes 38, 40, 42, and 44 to the sensing module within IMD 22.

In addition to providing cardiac pacing, IMD 22 may provide other electrical stimulation therapy, such as defibrillation, cardiac resynchronization, or cardioversion therapy. In this case, leads 28 and 30 may include additional electrodes. For example, one or both of leads 28 and 30 may include one or more elongated electrodes, which may, in some instances, take the form of a coil. IMD 22 may deliver defibrillation or cardioversion shocks to the heart via any combination of the elongated electrodes and housing 34, which may also function as an electrode.

In addition to more or fewer electrodes on leads 28 or 30, implantable medical system 14 may include more or fewer leads extending from IMD 22. For example, IMD 22 may be coupled to a third lead implanted within a left ventricle of heart 32 of patient 12. In another example, IMD 22 may be coupled to a single lead that is implanted within an atrium or ventricle of heart 32 of patient 12. As such, IMD 22 may be used for single chamber or multi-chamber cardiac rhythm management therapy. Additionally, leads 28 and/or 30 may not be implanted within heart 32 of patient 12, as is the case with epicardial leads. In other embodiments, IMD 22 may not be coupled to any leads, as is the case for a leadless pacemaker.

A patient having implanted medical system 14 may receive a certain therapy or diagnostic technique, surgery, or other procedure that exposes implantable medical system 14 to external fields, such as external fields 18 of FIG. 1. In the case of an MRI procedure, for example, implantable medical system 14 is exposed to the high frequency RF pulses and various magnetic fields described above to create image data regarding the patient 12. The RF pulses can induce currents within leads 28 and 30 of implantable medical system 14. The current induced in the leads 28 and 30 can cause certain effects, including heating, of the various lead components and/or tissue near the lead. Other medical procedures such as electrocautery procedures, diathermy procedures, ablation procedures, electrical therapy procedures, magnetic therapy procedures, or the like may also generate fields that interact with leads 28 and 30.

Leads 28 and/or 30 may include components or mechanisms to reduce or eliminate the amount of current induced by external fields. For example, implantable leads 28 and 30 may include an RF filter, RF trap, RF choke or other component located toward a distal end of the lead that blocks a large portion of the current induced by the high frequency RF fields from being conducted to tip electrodes 38 and 40 or ring electrodes 42 and 44. In another example, implantable leads 28 and 30 may include an RF shield to reduce the amount of current induced on leads 28 and 30. In a further example, implantable medical leads may include an RF shunt that shunts a large portion of the current induced on leads 28 and 30 away from the tip electrodes 38 and 40 to an energy dissipating surface. In still other examples, the conductors of leads may be designed with pitches, materials, turns, or other dimension or design to have a high inductance to reduce the amount of current that is induced on the lead.

However, whatever the component or mechanism included on the leads 28 and/or 30 to reduce or eliminate the amount of current induced by external fields, it is desirable to provide a physician and/or administrating personnel a visual assurance that leads 28 and/or 30, or the entire implantable medical system 14 is designed for safe application of a particular medical procedure, such as an MRI procedure. Radiopaque markers 46 may be placed on leads 28 and 30 to represent that implantable leads 28 and 30 and/or implantable medical system 14 is suitable for the particular medical procedure. Radiopaque markers 46 are visible on an X-ray or during fluoroscopy to provide a visual assurance that leads 28 and 30, or implantable medical system 14, is designed for safe application of the medical procedure of interest. In some instances, a signal or icon of radiopaque markers 46 may identify the implantable medical lead as being designed for safe application of a medical resonance imaging (MRI) procedure by a particular type of MRI device or under a particular set of MRI operating parameters. By individually tagging both leads 28 and 30, the administering personnel can be assured that both leads are safe for the given procedure.

Radiopaque markers 46 may, in some instances, be shaped to form a cylindrical lumen through which the lead to which it is associated passes through. Radiopaque markers 46 may simply be sleeves designed to identify the implantable medical lead as being designed for safe application of a medical procedure. In this case, lead 40 may also include a separate anchor sleeve. In other instances, radiopaque markers 46 may include other features to provide additional functionality, such as wings, suture grooves, or other mechanism to enable radiopaque markers 46 to be utilized as anchor sleeves.

Radiopaque markers 46 may be located in different locations along the length of lead 28 depending on whether the marker 46 is only an identification sleeve or has other functions. Radiopaque marker 46 associated with lead 28, for example, is located near the proximal end of lead 28 that connects to connector block 36. Radiopaque marker 46 associated with lead 30, on the other hand, is located at the site of exit of lead 30 from the vein through which it passes into the vasculature. Radiopaque markers 46 may be sized such that markers 46 are adequately visible via X-ray and fluoroscopy while being small enough to comfortably fit within or nearby the pocket near IMD 26, at the site of exit from the vein, or at another desirable location along leads 28 or 30. Radiopaque markers 46 may vary in size depending on the application for which it will be used or location along the leads 28 or 30.

Radiopaque markers 46 may be added to the respective leads during or after implantation of the lead in various ways including suturing, gluing, crimping, clamping, or other mechanism. Thus, if an implantable medical lead is later determined to be MR-compatible, the radiopaque marker may be added, such as at device replacement, to identify that the lead or system is designed for safe application of the medical procedure of interest. Moreover, by utilizing radiopaque markers 46, which are added as a sleeve, anchor or other separate component, there is no need to manufacture or construct the leads with the radiopaque marker being an integral part of lead. This would reduce manufacturing complexity and cost as well as reduce the size of the lead. A number of different examples of radiopaque markers are described herein.

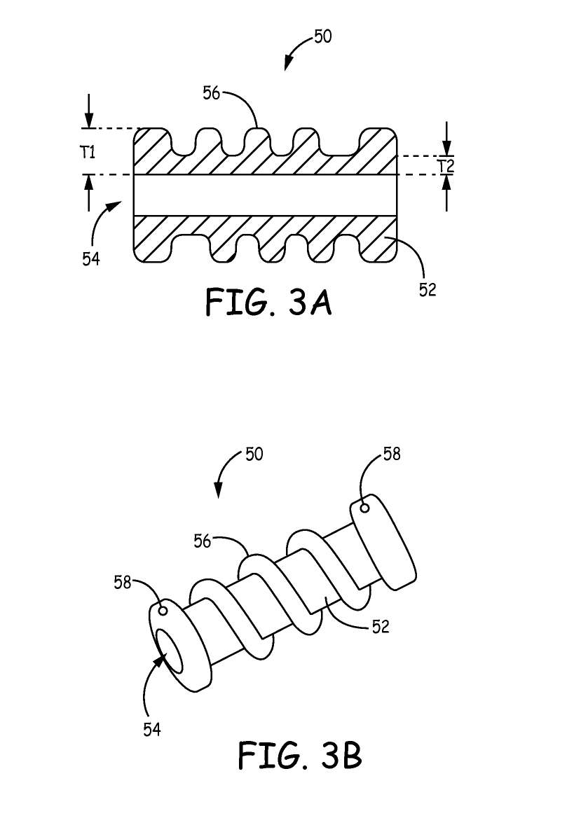

FIGS. 3A and 3B are schematic diagrams illustrating an example radiopaque marker 50 that may be connected to implantable medical leads to identify the leads as being designed for safe application of a medical procedure, such as an MRI procedure. Radiopaque marker 50 may correspond to one or both of radiopaque markers 46 attached to leads 28 or 30 of FIG. 2. Radiopaque marker 50 includes a body 52 being adapted to be disposed around a portion of an implantable medical lead. Body 52 forms a cylindrical lumen 54 through which a portion of the lead extends.

Body 52 is formed from a polymer material loaded with a radiopacifier such that radiopaque marker 50 is visible on an X-ray or during fluoroscopy. The polymer material used for body 52 may, for example, be silicone, polyurethane, PEBAX®, polyethylene, polypropylene, styrene block copolymers (SBC), PEEK, fluoroelastomers (such as PTFE, ETFE, PVDF-Polymer of vinylidene fluoride, tetrafluoroethylene (THV), hexafluoropropylene and vinylidene fluoride, and FEP), polysulfone, polyimide, acrylonitrile butadiene styrene (ABS), polymethylacrylates, polyvinyl chloride (PVC), polyamide, or a combination thereof. The radiopacifier may be bismuth (Bi), barium sulfate (BaSO4), tungsten (W), tungsten carbide, tantalum, titanium dioxide, platinum, niobium, palladium, or other radiopaque material, or combination thereof.

The loaded polymer may be mixed, blended or otherwise formed to have a light, medium or dark radiopacity. In some instances, it may be preferred that the loaded polymer have a light to medium radiopacity such that radiopaque marker 50 does not mask the conductors within the body of the lead. In other words, body 52 of radiopaque marker 50 has a radiopacity that is lighter than the conductors within the body of the lead to which radiopaque marker 50 is attached such that the portion of the conductors that lie under radiopaque marker 50 are also visible on an X-ray or during fluoroscopy. In this manner, any fracture in the portion of the conductor under radiopaque marker 50 may be identified by the X-ray or fluoroscopy. Moreover, by having a different radiopacity than the conductor, radiopaque marker 50 may not be mistaken for a lead fracture when one does not actually exist. The percentage of radiopacifier mixed with the polymer will of course depend on the type of radiopacifier used. A higher percentage by weight or volume of radiopacifier is needed when using barium sulfate than when using bismuth or tungsten to achieve the same radiopacity. In one example, the loaded polymer may comprise a silicone mixture loaded with 12.5% barium sulfate by volume.

Body 52 of radiopaque marker 50 includes areas of varying thicknesses. In the example illustrated in FIGS. 3A and 3B, body 52 includes portions have a first thickness (labeled T1 in FIG. 3A and referred to herein as the “thick portions”) and portions having a second thickness (labeled T2 in FIG. 3A and referred to herein as the “thin portions”). The portions of body 52 of radiopaque marker 50 will have different radiopacity based on the thickness of the loaded polymer. For example, the thick portions of body 52 appear more radiologically dense (i.e., have darker radiopacity) than the thin portions of body 52 in an X-ray or fluoroscopy or other imaging procedure.

In accordance with one aspect of this disclosure, body 52 of radiopaque marker 50 is formed such that the thick portions of body 52 form a symbol or icon 56 that identifies the implantable medical lead to which radiopaque marker 50 is attached as being designed for safe application of a medical procedure. In the example illustrated in FIGS. 3A and 3B, the thick portions of body 52 are formed into a coil-like symbol or icon that identifies the implantable medical lead to which radiopaque marker 50 is attached as being designed for safe application of a medical procedure. In some instances, the symbol or icon may be a symbol or icon representative of MR-conditionality of the leads to which radiopaque marker 50 is attached. This may be an industry-wide accepted symbol or icon or may be a symbol or icon associated with a particular company or product line.

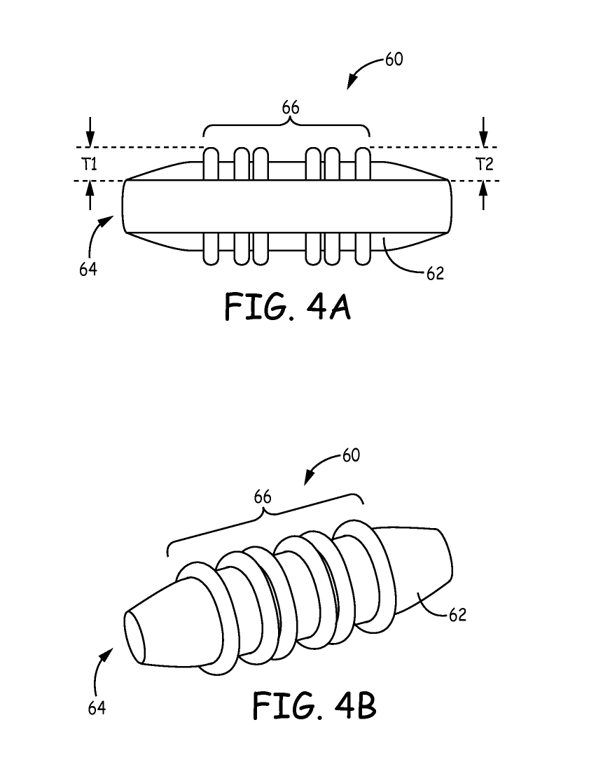

Although the symbol or icon 56 illustrated in FIGS. 3A and 3B is a coil-like symbol or icon, symbol or icon 56 may take on other shapes or designs. In some instances, the thick portions of body 52 may be formed into a symbol or icon made of a plurality of rings, dots, lines, or other structures or combination thereof that identifies the implantable medical lead as being designed for safe application of a medical procedure. In these cases, the administering personnel of the medical procedure may know to look for a particular pattern of rings, dots, lines, or other structures or combination thereof to identify the lead as being designed for safe application of a medical procedure. Such an embodiment is illustrated in FIGS. 4A and 4B. In other instances, thick portions of body 52 may be formed into one or more letters or numbers representative of the medical procedure to form the symbol or icon that identifies the implantable medical lead to which radiopaque marker 50 is attached as being designed for safe application of a medical procedure. For example, the thick portions of body 52 may be formed into “M R I” to indicate that the implantable medical lead to which radiopaque marker 50 is attached as being designed for safe application of an MRI procedure. In another example, the thick portions of body 52 may be formed into the letters/numbers “1.5 T MRI” or “3.0 T MRI” to indicate that the implantable medical lead to which radiopaque marker 50 is attached is designed for safe application of an MRI procedure by a particular type of MRI device, e.g., a 1.5 T MRI device or a 3.0 T MRI device, respectively.

As described above, body 52 of radiopaque marker 50 may be formed to define a lumen 54. The lead associated with radiopaque marker 50 may be routed through lumen 54 such that radiopaque marker 50 surrounds a portion of the lead. In other words, the lead to which radiopaque marker 50 is associated passes through lumen 54. Body 52 of radiopaque marker 50 may expand to a larger diameter than the lead such that radiopaque marker 50 may be positioned onto desired location of the lead. As described with respect to FIG. 2 above, the desired location may be near the proximal end of the lead, e.g., adjacent to the IMD, or located near the site of exit of the lead from the vein through which it passes into the vasculature. Body 52 of radiopaque marker 50 may expand to a larger diameter than the lead using a deployment tool to position the radiopaque marker 50 onto the lead. When removed from the deployment tool, body 52 contracts onto the lead to hold radiopaque marker 50 in place at the desired location.

In other instances, radiopaque marker 50 may include one or more features to aid in attaching radiopaque marker 50 at the location along the lead. Radiopaque marker 50 may, for example, be split along the longitudinal length such that radiopaque marker 50 may be placed on the lead without the use of deployment tool. Instead, the lead may be placed within the lumen via the lengthwise split and then attached or otherwise kept in place via the other attachment mechanisms. In one example, the other attachment mechanism may be one or more sutures that are placed in suture grooves or suture holes 58 of radiopaque marker 50. In another example, body 52 may be formed to include interlocking tabs, spring-loaded clip, or other connectors that may be closed, locked or otherwise connected after placing the lead within lumen 54 via the slit such that the lead remains within lumen 54. In a further example, body 52 may be formed to include wings or other protrusions such that radiopaque marker 50 may also be used as anchor sleeve at a desired location, such as at the site of exit of the lead from the vein through which it passes into the vasculature.

FIGS. 4A and 4B are schematic diagrams illustrating another example radiopaque marker 60 that may be connected to implantable medical leads to identify the leads as being designed for safe application of a medical procedure, such as an MRI procedure. Radiopaque marker 60 may correspond to one or both of radiopaque markers 46 attached to leads 28 or 30 of FIG. 2. Radiopaque marker conforms substantially to radiopaque marker 50 of FIGS. 3A and 3B, but the thick portions of body 62 of radiopaque marker 60 form a symbol or icon 66 that includes a plurality of rings along the longitudinal length of radiopaque marker 60 instead of a coil-like symbol or icon. Body 62 of radiopaque marker 60 may also define a lumen through which the lead passes through when attached to the lead. All of the attributes described above with respect to radiopaque marker 50 may be included within radiopaque marker 60.

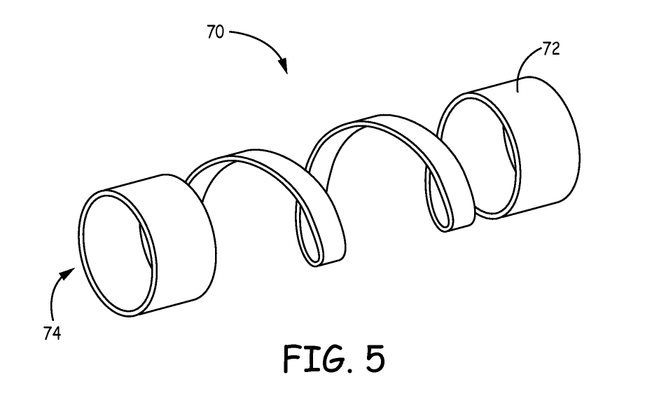

FIG. 5 is a schematic diagram illustrating another example radiopaque marker 70 that may be connected to implantable medical leads to identify the leads as being designed for safe application of a medical procedure, such as an MRI procedure. Radiopaque marker 70 may correspond to one or both of radiopaque markers 46 attached to leads 28 or 30 of FIG. 2. Radiopaque marker 70 includes a body 72 being adapted to be disposed around a portion of an implantable medical lead. Body 72 forms a lumen 74 through which a portion of the lead extends.

Body 72 may be a polymer material loaded with a radiopacifier such that radiopaque marker 70 is visible on an X-ray or during fluoroscopy. Suitable materials and mixtures are described above with reference to FIGS. 4A and 4B. Body 72 of radiopaque marker 70 is formed into a symbol or icon that identifies the implantable medical lead to which radiopaque marker 70 is attached as being designed for safe application of a medical procedure. In the example illustrated in FIG. 5, body 72 is formed into a coil-like symbol or icon that identifies the implantable medical lead to which radiopaque marker 70 is attached as being designed for safe application of a medical procedure. In some instances, the symbol or icon may be a symbol or icon representative of MR-conditionality of the leads to which radiopaque marker 70 is attached. However, unlike body 52 of radiopaque marker 50 of FIG. 3, which has areas of varying thicknesses, body 72 has a relatively uniform thickness and the entire body 72 forms symbol or icon.

As described above, body 72 of radiopaque marker 70 may be formed to define a lumen 74. The lead associated with radiopaque marker 70 may be routed through lumen 74 such that radiopaque marker 70 surrounds a portion of the lead. In other words, the lead to which radiopaque marker 70 is associated passes through lumen 74. Body 72 of radiopaque marker 70 may expand to a larger diameter than the lead such that radiopaque marker 70 may be positioned onto desired location of the lead. Body 72 of radiopaque marker 70 may expand to a larger diameter than the lead using a deployment tool to position the radiopaque marker 70 onto the lead. When removed from the deployment tool, body 72 contracts onto the lead to hold radiopaque marker 70 in place at the desired location. Although not illustrated in FIG. 5, radiopaque marker 70 may include one or more features to aid in attaching radiopaque marker 70 at the location along the lead, such as one or more suture grooves or suture holes, or wings or other protrusions that may be used to anchor radiopaque marker at the site of exit of lead 30 from the vein through which it passes into the vasculature.

Forming the entire body 72 as the symbol or icon may provide a coil-like structure made of a material that will not interact with the lead body of the associated lead to wear the lead body. Some radiopaque markers are constructed of a coil formed of wire, which in some instances, may wear, rub, or otherwise interact with the lead body of the associated lead. This in turn may have some undesirable consequences. Body 72 on the other hand has more attributes of a polymer and therefore is not as hard as a coil made from wire.

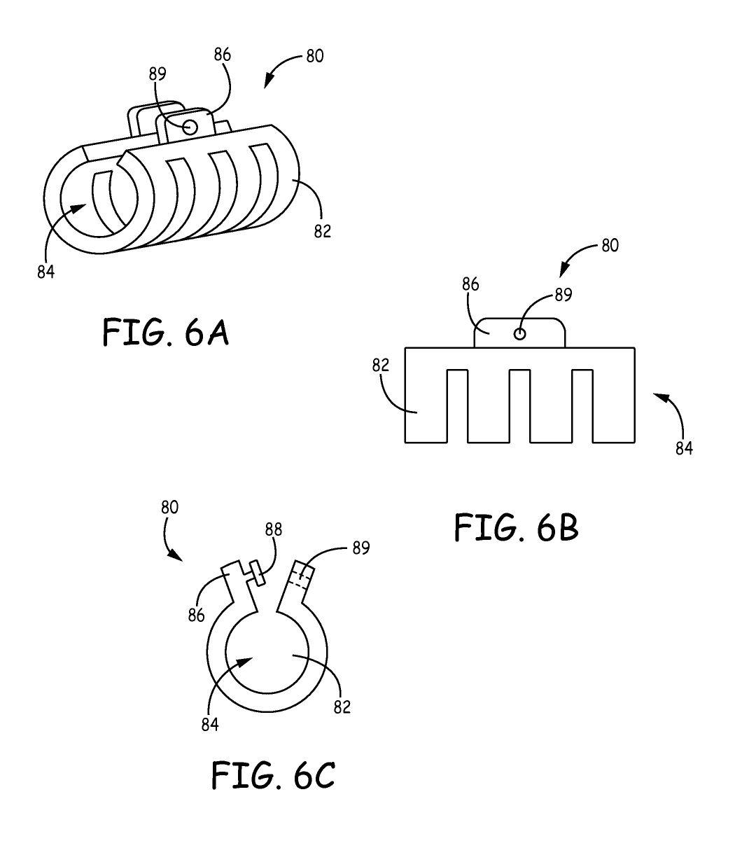

FIGS. 6A-6C are schematic diagrams illustrating another example radiopaque marker 80 that may be connected to implantable medical leads to identify the leads as being designed for safe application of a medical procedure, such as an MRI procedure. Radiopaque marker 80 may correspond to one or both of radiopaque markers 46 attached to leads 28 or 30 of FIG. 2. Radiopaque marker 80 includes a body 82 being adapted to be disposed around a portion of an implantable medical lead. Body 82 forms a lumen 84 through which a portion of the lead extends.

Body 82 may be a polymer material loaded with a radiopacifier such that radiopaque marker 80 is visible on an X-ray or during fluoroscopy. Suitable materials and mixtures are described above with reference to FIGS. 4A and 4B. Body 82 of radiopaque marker 80 is formed into a symbol or icon 86 that identifies the implantable medical lead to which radiopaque marker 80 is attached as being designed for safe application of a medical procedure. In the example illustrated in FIGS. 6A-6C, body 82 is formed into a plurality of ring-like structures that comprise the symbol or icon 86 that identifies the implantable medical lead to which radiopaque marker 80 is attached as being designed for safe application of a medical procedure. In this case, the administering personnel of the medical procedure may know to look for a particular pattern of ring-link structures to identify the lead as being designed for safe application of a medical procedure. In some instances, the symbol or icon 86 may be a symbol or icon representative of MR-conditionality of the leads to which radiopaque marker 80 is attached. Like body 72 of radiopaque marker 70 of FIG. 5, body 82 has a relatively uniform thickness and substantially the entire body 82 forms the symbol or icon.

As described above, body 82 of radiopaque marker 80 may be formed to define a lumen 84. The lead associated with radiopaque marker 80 may be routed through lumen 84 such that radiopaque marker 80 surrounds a portion of the lead. In other words, the lead to which radiopaque marker 80 is associated passes through lumen 84. Body 82 of radiopaque marker 80 of FIGS. 6A-6C is illustrated as including a slit along the length of body 82. The lead may be placed within lumen 84 via the slit, e.g., the slit may be expanded and place around the portion of the lead.

Body 82 includes a connection mechanism 86 that may be used to prevent the lead from exiting the lumen 84. Once the lead is placed within lumen 84, connection mechanism may be closed and possibly locked to keep the lead within lumen 84. In the example illustrated in FIGS. 6A-6C, connection mechanism 86 includes a tab 88 that extends through a hole 89 on the adjacent side of the connection mechanism to close the slit and keep the lead within lumen 84. In an alternate example, connection mechanism may extend along substantially the entire length of body 82 and include two or more tabs 88 and corresponding holes 89. In another alternate example, body 82 may include more than one connection mechanism 86, such as a first connection mechanism at one end of body 82 and a second connection mechanism at the opposite end of body 82.

In further instances, however, body 82 of radiopaque marker 80 may not have the connector mechanism described above. Instead, body 82 of radiopaque marker 80 may be attached or placed at the desired location using other techniques. In one example, body 82 may include one or more suture grooves or suture holes to aid in attaching radiopaque marker 80 at the location along the lead or wings or other protrusions that may be used to anchor radiopaque marker 80 at a desired location, such as at the site of exit of the lead from the vein through which it passes into the vasculature. In another example, body 82 may be an integral piece with no slit along the length of body 82 and may be expanded to a larger diameter than the lead, e.g. using a deployment tool, positioned onto desired location of the lead, and when removed from the deployment tool, body 82 may contract onto the lead to hold radiopaque marker 80 in place at the desired location.

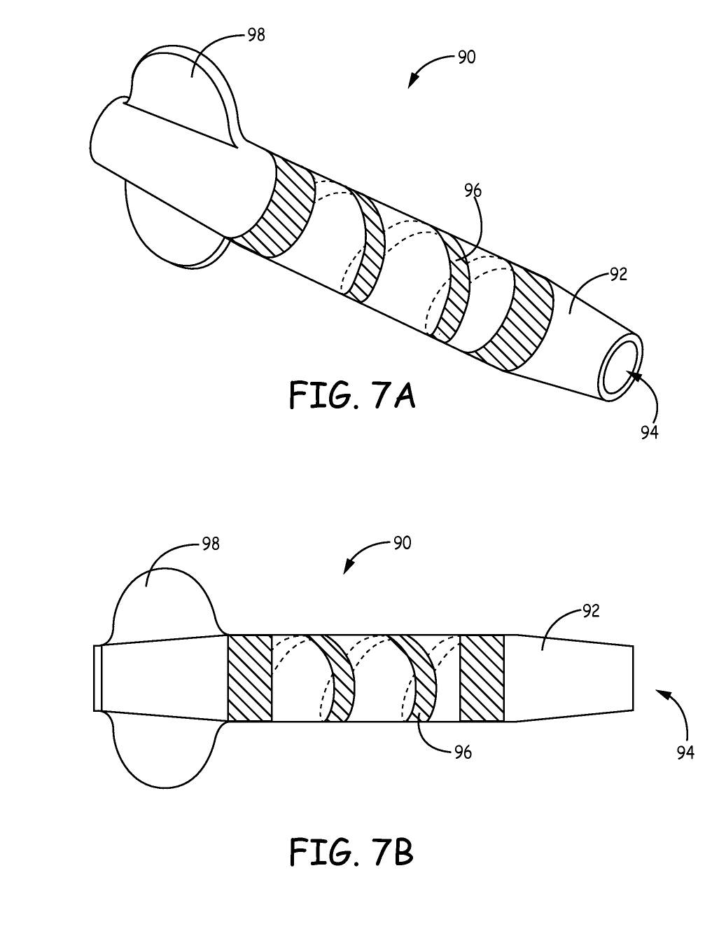

FIGS. 7A and 7B are schematic diagrams illustrating an example radiopaque marker 90 that may be connected to implantable medical leads to identify the leads as being designed for safe application of a medical procedure, such as an MRI procedure. Radiopaque marker 90 may correspond to one or both of radiopaque markers 46 attached to leads 28 or 30 of FIG. 2. Radiopaque marker 90 includes a body 92 being adapted to be disposed around a portion of an implantable medical lead. Body 92 forms a cylindrical lumen 94 through which a portion of the lead extends.

Body 92 may, in one embodiment, be formed from a polymer material, such as silicone, polyurethane, PEBAX®, polyethylene, polypropylene, styrene block copolymers (SBC), PEEK, fluoroelastomers (such as PTFE, ETFE, PVDF-Polymer of vinylidene fluoride, tetrafluoroethylene (THV), hexafluoropropylene and vinylidene fluoride, and FEP), polysulfone, polyimide, acrylonitrile butadiene styrene (ABS), polymethylacrylates, polyvinyl chloride (PVC), polyamide, or a combination thereof.

A symbol or icon 96 that identifies the implantable medical lead to which radiopaque marker 90 is attached as being designed for safe application of a medical procedure is added to body 92. Symbol or icon 96 may, in on example, be formed of a radiologically dense powder, such as a powder generated from bismuth (Bi), barium sulfate (BaSO4), tungsten (W), tungsten carbide, tantalum, titanium dioxide, platinum, niobium, palladium, or other radiopaque material. In another example, symbol or icon 96 may be formed of a radiologically dense liquid, such as intravenous contrast.

In one example, body 92 may be designed to include grooves in the shape of symbol or icon 96. The radiologically dense powder or tube of radiologically dense liquid may be placed in the grooves and covered (e.g., via overmolding or other technique) with additional polymer or other material. In this manner, the radiologically dense powder or liquid may form symbol or icon 96. In another example, body 92 may be designed to include a lumen and the radiologically dense powder or a radiologically dense liquid may be placed in the lumen to form symbol or icon 96.

In another example, symbol or icon 96 may be formed by sputtering, pad printing, inkjet printing, or otherwise dispensing a radiologically dense material onto body 92. In some instances, the radiologically dense material may be dispensed onto body 92 to form symbol or icon 96. In other instances, the radiologically dense material may be dispensed over some or all of body 92 and symbol or icon 96 may be formed by etching, laser cutting or otherwise removing portions of the radiologically dense material using subtractive manufacturing.

In other instances, the radiologically dense powder may be mixed, blended or otherwise combined with a polymer (such as the polymers listed above for body 92) to form radiopaque inserts in the shape of symbol or icon 96. The radiopaque inserts may be added to body 92 using any of a number of techniques. It may be desirable to have the radiopaque material not be in direct contact the body. In such a case, the polymer forming body 92 may be overmolded onto the radiopaque inserts to form radiopaque marker 90. In another example, the mixed polymer may be sandwiched between two polymer layers that form body 92. In other instances, the polymer mixed with the radiopacifier may be adhered to the outside of body 92 such that it is directly in contact with the body.

In the example illustrated in FIGS. 7A and 7B, the radiopaque inserts are formed into a coil-like symbol or icon. However, symbol or icon 96 may take on other shapes or designs. The symbol or icon may, for example, be made of a plurality of rings, dots, lines, or other structures or combination thereof that identifies the implantable medical lead as being designed for safe application of a medical procedure. In these cases, the administering personnel of the medical procedure may know to look for a particular pattern of rings, dots, lines, or other structures or combination thereof to identify the lead as being designed for safe application of a medical procedure.

In other embodiments, it may be desirable to also be able to visualize body 92 via X-ray or fluoroscopy. In such a case, the polymer forming body 92 may also be loaded with a radiopacifier such that body 92 is also visible on an X-ray or during fluoroscopy. The radiopacifier may be bismuth (Bi), barium sulfate (BaSO4), tungsten (W), tungsten carbide, tantalum, titanium dioxide, platinum, niobium, palladium, or other radiopaque material, or combination thereof. In this case, it is desirable to have body 92 be less radiopaque than the symbol or icon 96 that identifies the implantable medical lead to which radiopaque marker 90 is attached as being designed for safe application of a medical procedure such that there is enough contrast between body 92 and symbol or icon 96 to be visible on an X-ray or during fluoroscopy. For example, the polymer of body 92 may be mixed, blended or otherwise combined with the radiopacifier to have a light to medium radiopacity while symbol or icon 96 has a darker radiopacity.

Body 92 of radiopaque marker 90 may be formed to define a lumen 94. The lead associated with radiopaque marker 90 may be routed through lumen 94 such that radiopaque marker 90 surrounds a portion of the lead. In other words, the lead to which radiopaque marker 90 is associated passes through lumen 94. Body 92 of radiopaque marker 90 may expand to a larger diameter than the lead such that radiopaque marker 90 may be positioned onto desired location of the lead. As described with respect to FIG. 2 above, the desired location may be near the distal end of the lead, e.g., adjacent to the IMD, or located near the site of exit of the lead from the vein through which it passes into the vasculature. Body 92 of radiopaque marker 90 may expand to a larger diameter than the lead using a conventional deployment tool or custom deployment tool to position the radiopaque marker 90 onto the lead. When removed from the deployment tool, body 92 contracts onto the lead to hold radiopaque marker 90 in place at the desired location.

In other instances, radiopaque marker 90 may include one or more features to aid in attaching radiopaque marker 90 at the location along the lead. Radiopaque marker 90 may, for example, be split along the longitudinal length such that radiopaque marker 90 may be placed on the lead without the use of deployment tool. Instead, the lead may be placed within the lumen via the lengthwise split and then attached or otherwise kept in place via the other attachment mechanisms. In one example, the other attachment mechanism may be one or more sutures that are placed in suture grooves or suture holes 98 of radiopaque marker 90. In another example, body 92 may be formed to include interlocking tabs or other connectors that may be locked or otherwise connected after placing the lead within lumen 94 via the slit such that the lead remains within lumen 94. In a further example, body 92 may be formed to include wings or other protrusions that may be used to anchor radiopaque marker at a desired location, such as at the site of exit of lead 30 from the vein through which it passes into the vasculature.

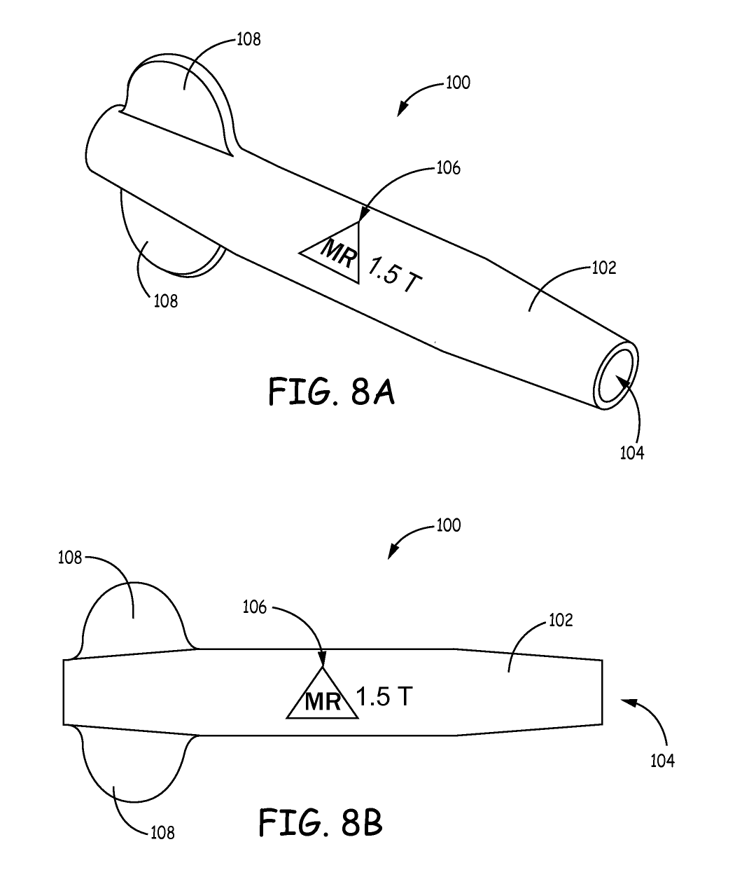

FIGS. 8A and 8B are schematic diagrams illustrating an example radiopaque marker 100 that may be connected to implantable medical leads to identify the leads as being designed for safe application of a medical procedure, such as an MRI procedure. Radiopaque marker 100 may correspond to one or both of radiopaque markers 46 attached to leads 28 or 30 of FIG. 2. Radiopaque marker 100 conforms substantially to radiopaque marker 90 of FIGS. 7A and 7B, but the symbol or icon 106 of FIGS. 8A and 8B is letters, shapes, or numbers representative of the medical procedure for which the lead is designed for safe application.

In the illustrated example, symbol or icon 106 is formed to an MR conditional symbol based on ASTM specification (a triangle enclosing the letter MR) as well as letters/numbers “1.5 T” to indicate that the implantable medical lead to which radiopaque marker 100 is attached is designed for safe application of an MRI procedure by a particular type of MRI device, e.g., a 1.5 T MRI device. In other instances, other widely accepted symbols may be included, such as the MR safe symbol based on ASTM specification which includes the letters MR enclosed in a square. Although illustrated as including an MR conditional symbol and as well as letter/numbers, symbol or icon 106 may include only the shapes, letters, and/or numbers representative of the medical procedure for which the lead is designed for safe application.

As described with respect to FIGS. 7A and 7B above, the symbol or icon that identifies the implantable medical lead to which radiopaque marker may be formed of a radiologically dense powder, such as a powder generated from bismuth (Bi), barium sulfate (BaSO4), tungsten (W), tungsten carbide, tantalum, titanium dioxide, platinum, niobium, palladium, or other radiopaque material, that is mixed, blended or otherwise combined with a polymer (such as the polymers listed) to form radiopaque inserts in the shape of symbol or icon 106.

Body 102 may, in one embodiment, be formed from a polymer material, such as silicone, polyurethane, PEBAX®, polyethylene, polypropylene, styrene block copolymers (SBC), PEEK, fluoroelastomers (such as PTFE, ETFE, PVDF-Polymer of vinylidene fluoride, tetrafluoroethylene (THV), hexafluoropropylene and vinylidene fluoride, and FEP), polysulfone, polyimide, acrylonitrile butadiene styrene (ABS), polymethylacrylates, polyvinyl chloride (PVC), polyamide, or a combination thereof. In other instances, body 102 may also be made from a polymer that is mixed with a radiopacifier. In this case, mixed polymer forming body 102 is mixed with a ratio of radiopacifier such that body 102 is less radiopaque than the mixed polymer forming symbol or icon 106 such that there is enough contrast between body 102 and symbol or icon 106 to be visible on an X-ray or during fluoroscopy. For example, the polymer of body 102 may be mixed, blended or otherwise combined with the radiopacifier to have a light to medium radiopacity while the polymer mixture forming symbol or icon 106 has a darker radiopacity.

Various examples have been described. These and other examples are within the scope of the following claims.

1. A radiopaque marker comprising:
a body formed of a polymer and being adapted to be disposed around a portion of an implantable medical lead;
a symbol formed of at least a radiologically dense powder added to the body and designed to identify the implantable medical lead as being safe application of a medical procedure.

2. The radiopaque marker of claim 1, wherein the symbol is designed to identify the implantable medical lead as being designed for safe application of a medical resonance imaging (MRI) procedure.

3. The radiopaque marker of claim 2, wherein the symbol is designed to identify the implantable medical lead as being designed for safe application of the MRI procedure by a particular type of MRI device.

4. The radiopaque marker of claim 1, wherein the symbol is designed to form a coil-like symbol.

5. The radiopaque marker of claim 1, wherein the symbol is designed to form to form a plurality of rings, dots, lines, or combination thereof on or within the body.

6. The radiopaque marker of claim 1, wherein the symbol is designed to form one or more letters or numbers representative of the medical procedure.

7. The radiopaque marker of claim 1, wherein the symbol is designed to form a symbol representative of MR-conditionality.

8. The radiopaque marker of claim 1, wherein the polymer of the body comprises at least one of silicone, polyurethane, PEBAX®, polyethylene, polypropylene, styrene block copolymers (SBC), PEEK, fluoroelastomers (such as PTFE, ETFE, PVDF-Polymer of vinylidene fluoride, tetrafluoroethylene (THV), hexafluoropropylene and vinylidene fluoride, and FEP), polysulfone, polyimide, acrylonitrile butadiene styrene (ABS), polymethylacrylates, polyvinyl chloride (PVC), polyamide, or a combination thereof.

9. The radiopaque marker of claim 1, wherein the body is formed of a polymer mixed with a radiologically dense material.

10. The radiopaque marker of claim 1, wherein the radiologically dense powder comprises at least one of bismuth (Bi), barium sulfate (BaSO4), tungsten (W), tungsten carbide, tantalum, titanium dioxide, platinum, niobium, palladium, or combination thereof.

11. The radiopaque marker of claim 1, wherein the symbol is formed of a polymer mixed with the radiologically dense powder.

12. The radiopaque marker of claim 1, wherein
the body is formed of a polymer mixed with a radiologically dense powder, and
the symbol is formed of a polymer mixed with a radiologically dense powder,
wherein the mixed polymer forming the symbol is radiologically denser than the mixed polymer forming the body.

13. The radiopaque marker of claim 1, wherein the body forms a lumen through which a lead can pass through the radiopaque marker.

14. The radiopaque marker of claim 13, wherein the body includes a slit along the longitudinal length of the body.

15. The radiopaque marker of claim 14, further comprising a mechanism to close at least a portion of the slit along the longitudinal length of the body to maintain the radiopaque marker at a location along a lead.

16. The radiopaque marker of claim 1, further comprising one or more anchoring mechanisms to aid in anchoring the radiopaque marker at a location within a body.

17. The radiopaque marker of claim 16, wherein the one or more anchoring mechanisms comprise one of suture holes, suture grooves, or suture wings.

18. A radiopaque marker comprising:
a body formed of a polymer and being adapted to be disposed around a portion of an implantable medical lead;
a symbol formed of at least a radiologically dense liquid added to the body and designed to identify the implantable medical lead as being safe application of a medical procedure.

19. The radiopaque marker of claim 18, wherein the body is formed to including a lumen in a shape of the symbol and the radiologically dense liquid is placed in the lumen to form the symbol.
